(19) United States
(12) Patent Application Publication
(10) Pub. No.: US 2013/0110059 A1
(43) Pub. Date: May 2, 2013

(54) PORTABLE FEMALE URINE COLLECTION DEVICE AND SYSTEM

(71) Applicant: KENNETH DAVID KOSSOW, HOLLYWOOD, FL (US)

(72) Inventors: KENNETH DAVID KOSSOW, HOLLYWOOD, FL (US); SHERRIE ANN UNVERSAW, HOLLYWOOD, FL (US)

(73) Assignee: KENNETH DAVID KOSSOW, HOLLYWOOD, FL (US)

(21) Appl. No.: 13/657,446

(22) Filed: Oct. 22, 2012

Related U.S. Application Data
(60) Provisional application No. 61/549,514, filed on Oct. 20, 2011.

(51) Int. Cl. A61F 5/455

(52) U.S. Cl. CPC A61F 5/455
USPC 604/329

(57) ABSTRACT

A female urine collection device, system and method are provided. The device includes a support device and a urine collection bag. The urine collection bag encapsulates the upper surface and sides of the support device, thus keeping the support device out of the path of the urine during urination. The support device is compact and sturdy so as to be capable of use and, if desired, of reuse.

### CROSS-REFERENCE TO RELATED APPLICATIONS

The present application claims priority to: co-pending Provisional Patent Application No. 61/549,514, filed on Oct. 20, 2011, entitled “PORTABLE FEMALE URINE COLLECTION DEVICE AND SYSTEM”; that application being incorporated herein, by reference, in its entirety.

### BACKGROUND OF THE INVENTION

1. Field of the Invention

The present invention relates to a low profile, compact, sanitary, washable, and child-friendly female urination collection and disposal device. Among other things, the device is useful for assisting women and female children with urinating, both inside and outside of the home, whether in a bathroom, a car, camping, or any other secluded location, and regardless as to whether there is a convenient source for immediate disposal, and also the special need for a device to assist with the collection of urine at doctors' offices, labs, clinics, etc.

2. Description of the Related Art

Female urination devices are primarily directed towards adult women with the objective of re-directing urine output to facilitate the ability to void (urinate) while standing-up. Often, such devices take the form of a funnel or other shaped device through which the urine is typically channeled into a toilet. The devices are typically compact and either reusable (but require cleaning after each use) or disposable. Female urination devices are also sometimes directed towards the collection and storage of urine, for example, in hospital settings.

One such device is shown in U.S. Pat. No. 5,571,095 to Ke-Way Lu (“Lu”). Lu discloses a disposable urine bag for females consisting of a bracket for carrying a polybag, a polybag for collecting urine, and a packing member fastened to the bracket to hold down the polybag. The bracket is described as a flat handle at one end and a bearing frame at the opposite end for fitting around the vulva. The bearing frame is said to have an endless mounting groove and what is described as a plurality of pointed blocks at the endless mounting groove, with the packing member being fitted into the endless mounting groove on the bearing frame to fix a water-proof bag in place, having a plurality of notches, which engage with the pointed blocks of the bearing frame the polybag. The polybag is described as having a plurality of spits around the border of its opening to allow the border of the bag opening to be turned inside out to ease the polybag fastening with the mounting groove of the bearing frame.

The Lu device, therefore, as illustrated constitutes a complex, disposable, multi-part assembly that must be assembled in interlocking fashion, i.e., with the packing member, strip, or rubber 2 fitted into the endless mounting groove 121 of the bearing frame 1, with the border of a polybag 3 having a plurality of splits around the border of its opening, to allow for entrapment between the two assemblies. Additionally, at least the packing member, strip, or rubber, which overlays the polybag, is necessarily in the direct stream of urine during use, the handle is not in any way shielded from urine, whether the occasional splash or otherwise, the packing member, strip, or rubber must be manually disengaged (after having been in the direct stream of urine flow) from the bearing frame and groove with the polybag and its plurality of splits and therefore connections to both the packing member, strip, or rubber, and the bearing frame must be carefully disengaged after use and notwithstanding the urine residue on the embodiments, and the polybag of collected urine carefully navigated without a sealing mechanism and with the required plurality of splits.

The Lu device is complicated, having a number of interconnected parts that must be carefully assembled to reduce the risk of leakage during use, with the further complexity and difficulty of disassembly of contaminated parts following use, disposal of the collected urine output collected in a polybag requiring a plurality of splits, and general inability to safely and securely hold the collected output for an extended period of time in the event that, for example, immediate disposal of the collected urine is not reasonably possible. The device is therefore not easily configured, disengaged, or otherwise susceptible to multiple uses, and would also require access to a washbasin or other facility to clean the various parts contaminated by the flow/splash of urine during use.

What is needed is a simple yet effective device to assist women, girls, and parents and caretakers of younger female children with urinating, both inside and outside of the home. What is additionally needed is a simple device to aid female urination and urine collection that does not require interlocking parts for assembly and disassembly. What is further needed is a device to aid female urination and urine collection which may be used and reused with a minimum of effort and without having to disengage interlocking parts that have been in the urine flow path.

### SUMMARY OF THE INVENTION

The present invention is directed to a novel low profile, compact, sanitary, washable, efficient, and child-friendly female urine collection and disposal device, system and method. In one particular embodiment of the invention, the urine collection and disposal device includes a single support unit with a polyethylene zipper-style collection bag that, when configured, encapsulates the device so as to avoid direct skin contact or contamination with urine during the urination/voiding process. The device may, therefore, be easily assembled within seconds, used without risk of contamination or leakage (with the urine held in an enclosable zipper-style collection bag, if desired), and immediately reused (without risk of cross-contamination in the case of multiple users) or stored for later use without any need for cleaning. In another particular embodiment, the device has a low profile design, so as to be readily carried or stored in a shirt pocket, purse, fanny pack, glove box, bicycle pouch, or other compact compartment for easy and convenient access.

The device provides a simple, convenient, compact, and cost-effective tool to primarily assist parents and caretakers of toddlers and children with voiding (urinating) outside of the home especially where bathroom facilities are either not readily available (such as in remote locations such as while hiking, camping, fishing, boating, swimming, flying, on long car trips, etc.) or, as is often the case, not clean or are otherwise undesirable (such as while shopping and at fairs, festivals, and sporting events), and in cases where the child, for example, may not have sufficient motor skills, dexterity, patience, understanding, or general comfort-level required to use other more elaborate and complicated devices such as, for example, the cone and other configurations. The device will also comfortably fit a small child as well as a large adult woman's anatomy, and may therefore be readily used by any female who may be in need of assistance with urinating, both inside (such as elderly or disabled women) and outside of the home, and also as a tool for facilitating the collection of urine specimens at doctors' offices, labs, clinics, etc.

In one particular embodiment of the invention, the device is equipped with a small handle that enables the user, whether an adult woman, a child, or a parent or caretaker in the case of toddlers and young children, to easily position the device in close proximity to the urethra without the device ever having any direct personal contact (due to its being encased by the collection bag), and without the parent or caretaker having any personal contact with the child's genitalia.

Other features, which are considered as characteristic for the invention, are set forth in the drawings and the appended claims.

Although the invention is illustrated and described herein as embodied in a portable female urine collection device and system, it is nevertheless not intended to be limited to the details shown, since various modifications and structural changes may be made therein without departing from the spirit of the invention and within the scope and range of equivalents of the claims.

The construction of the invention, together with additional objects and advantages thereof, will be best understood from the following description of the specific embodiment when read in connection with the accompanying drawings.

### BRIEF DESCRIPTION OF THE DRAWINGS

For a fuller understanding of the nature of the present invention reference should be made to the following detailed description taken in connection with the accompanying drawings in which:

FIG. 1 is a top plan view of a portable female urine collection device in accordance with one particular embodiment of the present invention and FIG. 1A is a cross-sectional view taken in the direction of line A-A′ of FIG. 1.

FIG. 1B is an isometric view taken from the top of the portable female urine collection device of FIG. 1.

FIG. 2 is bottom plan view of a portable female urine collection device in accordance with one particular embodiment of the present invention.

FIG. 2A is a cross-sectional view taken in the direction of line B-B′ of FIG. 2 of a portable female urine collection device in accordance with one particular embodiment of the present invention.

FIG. 2B is an perspective view taken from the bottom of a portable female urine collection device in accordance with one particular embodiment of the present invention.

FIG. 3 is a perspective view of a portable female urine collection device in accordance with one particular embodiment of the present invention with a collection bag being joined therewith.

FIG. 4 is a side plan view of a portable female urine collection device in accordance with one particular embodiment of the present invention having a collection bag applied over the device.

FIG. 5 shows a portable female urine collection kit in accordance with one particular embodiment of the invention.

### DETAILED DESCRIPTION OF THE PREFERRED EMBODIMENT

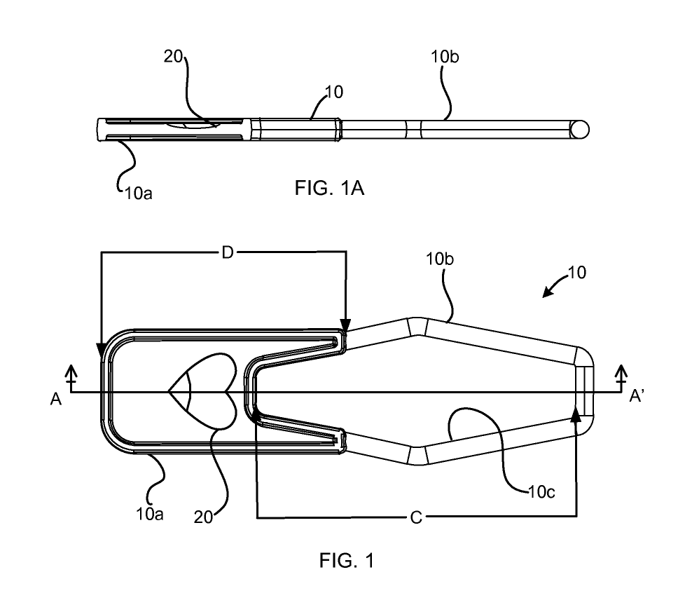

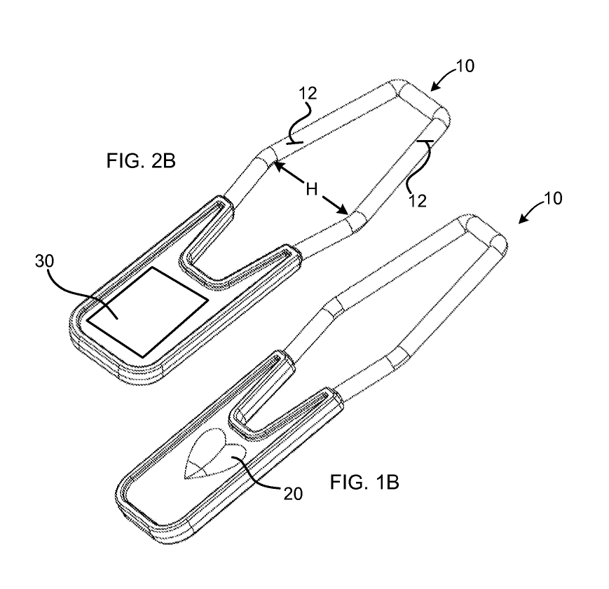

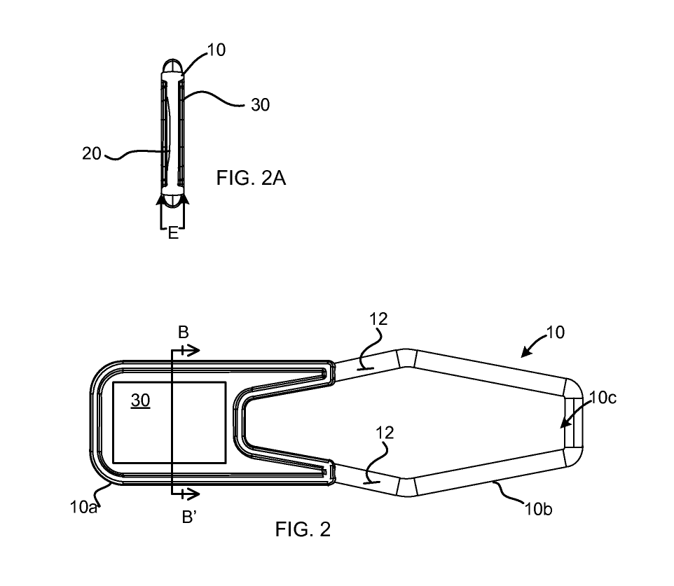

Referring now to FIGS. 1-4, there is shown one particular embodiment of a portable female urine collection device 50 including a support device 10 in accordance with the present invention. The support device 10 is contoured to extend, from the front, comfortably between the legs of a female to collect urine in a bag disposed over the support device 10. In one particular embodiment, the support device 10 is 140 millimeters long and 5 millimeters in thickness, although this is not meant to be limiting, as other lengths and thicknesses of the support device 10 may be used.

The support device 10 is an elongate bracket including a handle or handle portion 10a and a receiving receptacle portion 10b, defining a receiving aperture or opening 10c. In one particular embodiment of the device, the support device 10 is flat, such that the handle and receiving portion are disposed entirely within the same plane. This ensures that the device 10 is compact, thus making it easily portable in a shirt pocket, purse, fanny pack, glove box, bicycle pouch, or other compact compartment for easy and convenient access. The overall length and width of the receiving receptacle portion 10b and, correspondingly, of the receiving aperture 10c, can vary. However, in one particular embodiment of the invention, the overall length “C” of the receiving aperture 10c, defined inside of the receiving receptacle portion 10b, is approximately 90 millimeters, with the widest point of the aperture being approximately 45 millimeters from each inner surface of the receiving receptacle portion 10b. In one particular embodiment of the invention, the receiving receptacle portion is about 42-43 millimeters wide at its widest point.

The support device 10 has a relatively small handle 10a, although the length, width, and thickness could vary, as desired. In one particular embodiment of the invention, the length “D” of the handle is approximately 70 millimeters in overall length, with approximately 25 millimeters providing additional support for the receiving receptacle portion and approximately 45 millimeters to grip the device. In this particular embodiment of the invention, the handle is approximately 35 millimeters in width, and 5-6.5 millimeters in thickness, thus providing for a device 10 that has increased compactness, strength, durability, and portability. In another particularly preferred embodiment, the handle has an approximate thickness “E” of 6.35 millimeters.

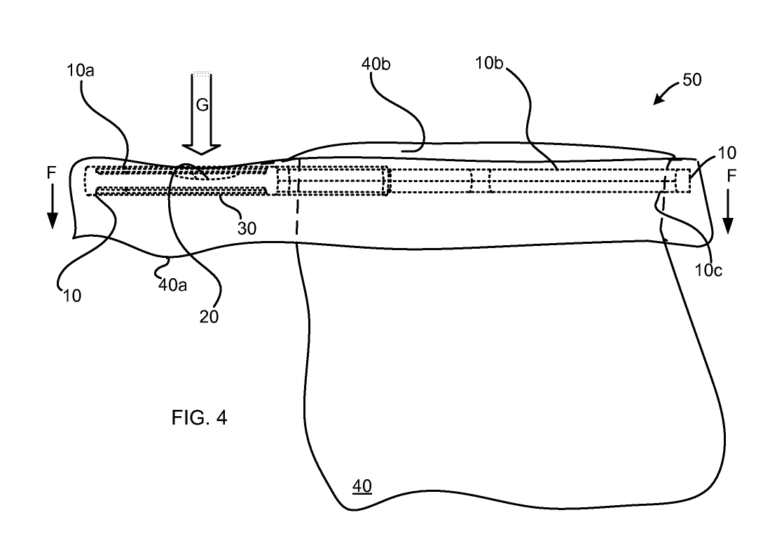

In the present preferred embodiment, the handle 10a also has a slight indentation 20 on the top surface of the handle portion 10a. In one particular embodiment of the invention, the indentation 20 is approximately 1.3 millimeters deep. The indentation 20 is contoured to receive the tip of the thumb (in the direction of arrow “G” of FIG. 4), while the remainder of the same hand of a user grasps the handle 10a, with the users remaining fingers contacting the bottom surface of the handle 10a. When used with a bag, the holder of the device entraps a portion of the bag in the indentation 20 with the tip of the holder's thumb, thus applying a tension to the bag to oppose the pulling force on the bag created by the bag filling with urine. Optionally, the bottom surface of the handle 10a can include a logo formed thereon or an indentation 30 for receiving a label or silkscreen containing a logo.

As can be seen from the figures, in the presently described embodiment, the end of the receiving receptacle portion 10b distal from the handle 10a is less wide than the width of the handle 10a. For example, in one particular embodiment of the invention, the handle 10a is approximately 35 millimeters, while the distal end of the receiving receptacle portion 10b is approximately 20 millimeters, and the width “H” of the aperture in the receiving receptacle portion 10b, at its widest point is approximately 32 millimeters. This is not meant to be limiting, however, as the distal end of the receiving receptacle portion 10b can be any desired width. In another preferred embodiment of the invention, the distal end of the receiving receptacle portion 10b, narrows to a diameter of approximately 15 millimeters on the inside and 25 millimeters on the outside taking into account the thickness of the lateral supports 12 of the receiving receptacle portion 10b, with the lateral supports 12 being joined at the distal end by a curved perpendicular support. The narrowing and curvature of the distal end is intended for increased comfort between the legs of all users, including, but not limited to, toddlers and children. More particularly, the unique design (shape) of the lateral supports 12 of the receiving receptacle portion 10b facilitates placement of the device while eliminating discomfort. The lateral supports 12 additionally permit quick and easy placement, removal and/or replacement of a low cost, disposable collection bag 40. In one particular embodiment of the invention, the bag 40 completely encapsulates the support device 10 and thereby keeps the support device 10 clean of all urine and other bodily contact and/or discharge.

Additionally, the end of the handle 10a proximal to the receptacle receiving portion 10b could be selected to be any desired size, and in one particular embodiment of the invention, includes a cutout of the handle, which helps form a portion of the receiving aperture 10c. The end length of the receiving aperture 10c is, in one particular embodiment, approximately 12 millimeters wide at the base of the cutout portion of the handle. Thus, in one particular embodiment of the invention, the handle end of the support device 10 is approximately 35 millimeters wide, the widest portion of the receptacle-receiving portion is 42 millimeters wide, and the distal end of the receptacle-receiving portion is approximately 20 millimeters wide. Consequently, it can be seen that the distal end of the device is tapered for comfort, while the receptacle aperture 10c (bordered by the handle at one end of the support device 10, and by a perpendicular support at the other end) is sized to conveniently and efficiently collect the urine voided from the urethra.

The support device 10 may be manufactured out of a variety of materials, including, but not limited to, polypropylene, aluminum, and steel. In one particularly preferred embodiment of the invention, the support device 10 is made as a unitary piece of polypropylene in an injection molding process. Alternately, as desired, the support device 10 may be formed using one or more pieces and/or other materials, and/or by different processes, such as milling, extruding, punch cutting, three-dimensional printing, etc. Optionally, the support device 10 may be made or painted a variety of colors and/or have other decorative elements, so as to be visually appealing and/or child-friendly. Using a unique coloring or decoration can be child friendly and facilitate the child's general embracing of the device.

Referring now to FIGS. 1-5. The support device 10 is intended to be used with a collection bag 40 capable of holding urine without leaking. In one particular embodiment of the invention, the bags 40 are manufactured out of polyethylene or another relatively lightweight material and may be bio-degradable, if desired, for ultimate disposal in the nearest available facility. In one particularly preferred embodiment of the invention, the bags 40 include a zipper style closure, although other types of bags may be used. Additionally, while the bags may be of varying widths and lengths/depths, in one particular preferred embodiment of the invention, the dimensions are approximately 165 millimeters in width and approximately 190 millimeters in length/depth with a thickness of approximately 2 millimeters, which is sufficient volume and strength to hold the maximum estimated urine output of 500 milliliters weighing approximately 16 ounces. Additionally, it was found that commonly available standard-sized sandwich bags worked well with one particularly preferred embodiment of the invention. In one particular embodiment of the invention, the collection bag 40 is sized to cover the entire top surface of the support device 10 and to tightly engage the outer peripheral edge (i.e. sidewall) of support device 10 in a frictional fit, when folded over the top and side edge of the support device 10, as shown more particularly in FIG. 4. In one particular embodiment of the invention, the support device 10 has been particularly sized to work with standard sized lunch or sandwich bags, as well as deeper custom bags. This conveniently allows consumers to use bags that are commonly available and found at their local supermarket as replacements for the bags 40.

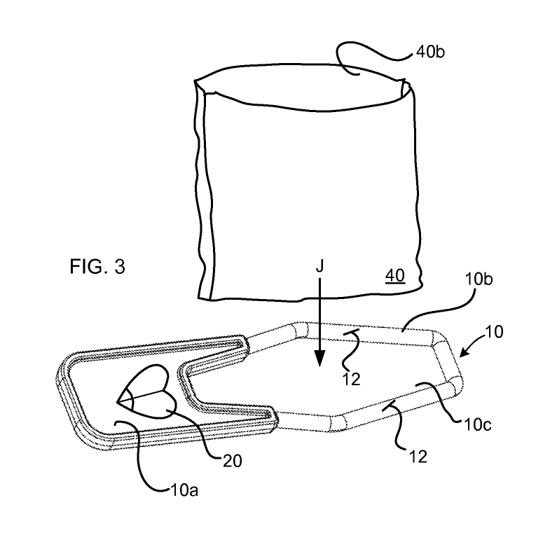

Referring more particularly to FIGS. 3 and 4, there is shown a device 10, in accordance with one particular embodiment of the invention, wherein a bag 40 is inserted through, and/or mated with, the support device 10 to form a urine collection device 50. More particularly, the support device 10 is held with the topside up (i.e., with the indentation 20 facing upward) and the bottom portion of the bag 40 is inserted through the receptacle aperture 10c from the top, i.e., in the direction of arrow “J”. Note that the entire bag 40 is not passed through the supports 12 of the receptacle receiving portion 10b, but rather, a lip portion 40a at the top, opening end 40b of the bag 40 is folded over, in the direction of arrows “F” of FIG. 4, over the entire device, as shown in FIG. 4. Additionally, the urine collection bag 40 encapsulates the entire upper surface and side portions of the support device 10, leaving no part of the support device 10 above the bag or in the path of the flowing urine. The person holding the support device 10 grasps the support device 10 with the bag opening 40b at the top, by wrapping their hand around the handle 10a, with a portion of the top of the bag 40a grasped between the palm and fingers of the holder's hand and the handle 10a. Additionally, simultaneously, the tip of the holder's thumb is pressed against the indentation 20 on the top surface of the handle 10a, to further retain the folded lip 40a of the bag 40 securely over the entire device 10, without slipping. The holder holds the urine collection device 50 such that the opening of the bag 40b is retained under the urethra of the user during urination. After urination, the bag 40 is emptied of urine in a convenient location and the folded-over lip 40a of the bag 40 is unfolded to remove the bag 40 from the aperture 10c. For example, the urine-containing bag 40 can be emptied into the nearest toilet, and then properly disposed of by removing the bag 40 from the top or bottom of the support device 10 and discarding the bag 40, while retaining the support device 10. Alternately, in certain circumstances the bag 40 may include a zipper type closure device so that a bag 40 containing urine can be sealed for later disposal (for example, if used in a car or other remote location where immediate disposal is not practical). Other types of sealing mechanisms than zipper type can be used, including, but not limited to adhesive strips, foldover lips, twist ties and other available sealing mechanisms. Additionally, the support device 10 should be manufactured to have sufficient structural integrity for use and, if desired, for reuse, and, in one particular embodiment, may be made to be dishwasher safe.

It has been found that the urine collection device 50 works appropriately with the bag held merely by a friction fit caused by the tight fit of the bag 40 folded over the support device 10 and held by the hand of the person holding the support device 10, and thus, does not need any further mechanism disposed over the bag to retain the bag in place. The support device 10 has been tested with standard sized sandwich bags having a zipper-type closure mechanism. The inclusion of such a zipper-type closure mechanism has been found to add to the structural integrity of the bag 40, and to aid in the friction fit with the support device 10. The bags, when folded over the lateral and distal supports, effectively and completely encapsulate the entire top and peripheral sidewalls of the device, helping support and maintain the bag in proper position for collection of urine with minimal effort, and also shielding the device from direct contact between the person using the device and/or any bodily fluids. By retaining the bag over the device in this way, no reusable portion of the device comes in contact with the stream of urine. Additionally, as no further device is needed in order to retain the bag in the aperture 10c, the urine collection device 50 (i.e., the support device 10 in combination with the bag 40) is extremely simple to assemble, more so than the device of Lu discussed above, and more simple to disassemble and reuse than that of Lu. However, if desired (although not necessary) the bag 40 may be clipped or otherwise attached to the lateral supports 12 and/or distal supports.

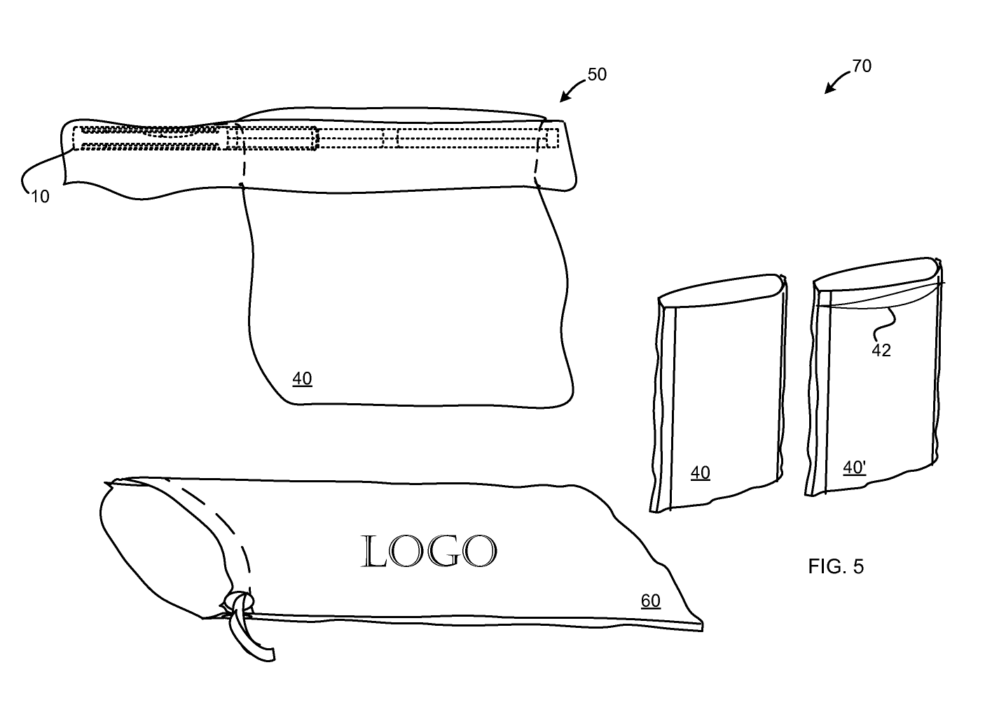

Referring now to FIG. 5, there is shown a kit 70 including a urine collection device 50 having a support device 10, as described in connection with FIGS. 1-4. The kit 70 may be provided with a bag 10 already mounted on the support device 10, or separately, as desired. Additional bags 40 can be provided as part of the kit 70, to enable reuse of the support device 10. Additionally, in any of the embodiments described herein, the bag 40 may be, or may be replaced by, a bag 40′ having an interlocking zipper type closure 42, such as is made under the ZIPLOC trademark. In one particular embodiment of the invention, the collection bag 40′ is approximately 2 millimeters thick, 165 millimeters wide and approximately 190 millimeters deep from the zipper mechanism 42 to the bottom of the bag 40′.

If desired, the device 10 and bags 40, 40′ can be provided in a re-closable, reusable carry bag 60. Re-closable, reusable carry bag 60 can be a nylon, vinyl, microfiber or polypropylene sleeve with an internal pocket, or a small case sized to hold the support device 10 along with several bags and possibly tissues for use with the support device 10. If desired, the bag 60 can additionally carry a decorative indicia or logo. The compact, low-profile size and shape of the support device 10, packed with several bags into the small case, bag or sleeve 60, is intended to facilitate portability of a urine collection device 50 and its storage in a shirt pocket, purse, fanny pack, glove box, bicycle pouch, or other compact compartment for easy and convenient access. Alternately, if desired, the device 10 and bags 40 can be sold together as a kit in or with other forms of packaging, such as in a blister pack or bag and hang tag, or in no packaging at all.

As can be seen from the description herein, the invention provides a compact, very portable, reusable urine collection device that is easy to assemble, use and reuse, and is hygienic in its use and reuse. The device is particularly suited for use by adult women, girls, and parents and caretakers of younger female children who are in need of a simple yet effective device to assist with urinating, both inside and outside of the home, and also the collection of urine at doctors' offices, labs, clinics, etc. Additionally, the device does not require a complex multi-part assembly, may be used and reused repeatedly with little effort or risk of leakage, without contamination or required access to a washbasin or other cleaning facility, and with the ability to store the collected urine output without risk of leakage.

Accordingly, while a preferred embodiment of the present invention is shown and described herein, it will be understood that the invention may be embodied otherwise than as herein specifically illustrated or described, and that within the embodiments certain changes in the detail and construction, as well as the arrangement of the parts, may be made without departing from the principles of the present invention as defined by the appended claims.

We claim:

1. A urine collection device, comprising:
a support device including a handle and receiving receptacle portion defining an aperture;
a collection bag; and
at least a portion of said collection bag being retained through said aperture and another portion of said collection bag is folded over an upper surface and outer peripheral edge of said support device.

2. The device of claim 1, wherein the support device is flat.

3. The device of claim 1, wherein said another portion of said collection bag is folded over the entire upper surface and peripheral edge of said support device.

4. The device of claim 3, wherein the collection bag is sized to tightly engage the outer peripheral edge of support device in a friction fit when folded over said support device.

5. The device of claim 1, wherein an upper surface of said handle includes an indentation sized to receive a tip of a thumb therein.

6. The device of claim 1, wherein said collection bag includes a zipper type closure mechanism.

7. The device of claim 1, wherein said support device is tapered such that said retainer receiving portion tapers outward from said handle, the width of said retainer receiving portion at its widest point being wider than the greatest width of said handle, said retainer receiving portion tapering in from said widest point to an end of said retainer receiving portion distal from said handle.

8. The device of claim 7, wherein a width of said retainer receiving portion at its end is less than the width of said handle.

9. A support device for use in a female urine collection device, the support device comprising:
a handle and retainer receiving portion defining an aperture;
the support device being flat, with the handle and retainer receiving portion being disposed entirely in the same plane.

10. The support device of claim 9, wherein an upper surface of said handle includes an indentation sized to receive a tip of a thumb therein.

11. The device of claim 9, wherein said support device is tapered such that said retainer receiving portion tapers outward from said handle, the width of said retainer receiving portion at its widest point being wider than the greatest width of said handle, said retainer receiving portion tapering in from said widest point to an end of said retainer receiving portion distal from said handle.

12. The device of claim 11, wherein a width of said retainer receiving portion at its end is less than the width of said handle.

13. A kit for aiding in female urine collection, comprising:
a support device according to claim 9;
a plurality of collection bags sized to be used with said support device.

14. The kit of claim 13, further comprising, a reusable carry bag including the support device and the plurality of collection bags.

15. A kit for aiding in female urine collection, comprising:
the device according to claim 1;
a reusable carry bag including the support device and the plurality of collection bags.

16. The kit of claim 15, further comprising a plurality of collection bags.

17. A method of using a urine collection device, comprising the steps of:
providing a urine collection device according to claim 1;
holding the urine collection device with the collection bag entrapped between a hand of the user and the handle of the support device; and
during the holding step, collecting urine in the portion of the collection bag disposed through the aperture.

18. The method of claim 17, further comprising the step of reusing at least the support device in the further collection of urine.

19. The method of claim 18, wherein said reusing step includes the steps of:
disposing of the collected urine and of the collection bag used to collect the urine;
mating a further collection bag with the support device; and
collecting urine in a portion of the new collection bag.

20. A method of using a urine collection device, comprising the steps of:
providing a support device according to claim 9;
disposing a collection bag over the support device such that at least a portion of the collection bag is retained through the aperture and another portion of the collection bag is folded over an upper surface and outer peripheral edge of the support device;
holding the support device with the collection bag entrapped between a hand of the user and the handle of the support device; and
during the holding step, collecting urine in the portion of the collection bag disposed through the aperture.
